(19) United States
(12) Patent Application Publication
(10) Pub. No.: US 2013/0331755 A1
(43) Pub. Date: Dec. 12, 2013

(54) ORTHOTIC LUMBAR SUPPORT BELT

(71) Applicant: Ellen Rotblatt, Farmington Hills, MI (US)

(72) Inventor: Ellen Rotblatt, Farmington Hills, MI (US)

(21) Appl. No.: 13/490,038

(22) Filed: Jun. 6, 2012

(51) Int. Cl. A61F 5/00

(52) U.S. Cl. 602/19

(57) ABSTRACT

The present invention relates to an orthotic lumbar support garment intended to be worn under a patient's clothing and affixed around the abdomen of the patient generally positioned above the patient's hips. The orthotic lumbar support garment of the present invention is in the form of a belt-like apparatus which is configured to prevent, restrict or reduce the motions which would aggravate various spinal conditions existing in a patient, such as spondylolithesis (LS), spondylosis, or a slipped disk condition, by applying lateral pressure to the spine from the abdomen through conformable chambers disposed on the belt adjacent the patient's abdomen.

### BACKGROUND OF THE INVENTION

The present invention relates to an orthotic lumbar support garment intended to be worn under a patient's clothing and affixed around the abdomen of the patient generally positioned above the patient's hips. The orthotic lumbar support garment of the present invention is in the form of a belt-like apparatus which is configured to prevent, restrict or reduce the motions which would aggravate various spinal conditions existing in a patient, such as spondylolithesis (LS), spondylosis, or a slipped disk condition.

Currently, patients in need of spinal braces or other orthopedic devices can choose between devices which are either custom-made for the individual patient, or are themselves customizable. A customizable orthotic device is a device which uses premanufactured modular components that allow a patient to customize the device according to the individual needs of the patient. These customizable devices are generally less expensive and readily available to a prescribing physician as compared to a custom-made orthotic device. Inherent disadvantages with a customizable device include a sacrifice in patient comfort level in return for the modularity of the device. That is, the customizable device is a premanufactured device without the individual characteristics of the patient being considered. This lack of individualization can lead to an ill-fitting device which has customizable features, yet may still include premanufactured modular components that do not fit an individual patient correctly. Furthermore, due to the modular nature of a partially customizable device, such devices do not offer the most ideal corrective support, prevention, or overall medical benefit to an individual patient. The other choice available to patients in need of spinal braces and orthopedic devices are custom-made devices which are intended and manufactured for one particular patient in accordance with the specific medical conditions of that patient. These devices are generally much more expensive than a customizable premanufactured device but offer superior comfort and an overall increased medical benefit to the patient.

Spinal support and bracing devices available currently are generally bulky devices which can comprise large rigid pieces of support material incorporated therein. These rigid pieces of material, given their size and inability to conform to an individual patient, prevent the patient from comfortably wearing the device for long periods of time. Further, the large construction of these devices also prevents a patient from inconspicuously wearing the device under the patient's clothing. Devices which are capable of being worn under a patient's clothing are also known, but are generally highly constrictive. These devices, which can be worn under a patient's clothing, generally substitute the highly rigid materials of other inconspicuous devices for a more compressive girdle-like fit.

Generally, traditional spinal orthopedic devices are designed to be static and prevent a patient's motion in most, if not all, directions. These devices do not discriminate between motions which are harmful to the patient's condition and motions which, in fact, would be beneficial to the patient. The direction, duration, and intensity of various motions of a patient are dictated by the patient's spinal condition, and a greater degree of freedom, as well as quality of life, can be realized when an orthopedic device which allows for increased patient mobility and comfort is utilized. Currently known orthotic lumbar support devices typically create anterior loads on a patients vertebrae which is counter indicated for conditions such as spondylolithesis.

Thus, a need exists for a device which is custom fit to the patient for comfort and overall medical benefit, yet modular enough to reduce costs. Further, the device should be low profile, such that it can be worn under the patient's clothing while not overly constricting the patient's movement. Further, an orthotic device which would sense and adapt to the patient's own motions would provide further medical benefits to the device user. If an orthotic device could detect the preparation of a patient's motion, determine if that motion would be detrimental to the patient and further be intelligent enough to preempt the motion if, in fact, such motion would be detrimental, by restricting, limiting, or immobilizing the patient from inducing such motion, a patient's overall wellbeing is better cared for. Similarly, if a patient's motion would, in fact, be beneficial to the patient's spinal condition, the device would ideally sense that this is, in fact, the case and allow the patient to move freely in such a manner while still supporting the patient.

### SUMMARY OF THE INVENTION

The present invention is considered a smart orthopedic garment in the form of an orthotic lumbar support belt which is composed of a static belt and an adaptive subsystem. The garment is designed to be low-profile such that it can be worn under a patient's clothing and fitting to the patient's abdomen while being contoured to the patient's pelvic bone for comfort and alignment. While other orthotic support devices typically produce anterior loads, the present invention is designed to restrict anterior movement of a patient's vertebrae.

One aspect of the present invention includes an orthotic spinal support device for controlling movement of a patient, comprising a belt apparatus having a belt body portion adapted to couple to a patient adjacent a spinal region of the patient. The belt apparatus includes at least one conformable chamber or bladder configured to apply lateral pressure to predetermined areas of an abdomen of the patient to limit anterior movement of a patient's vertebrae. A plurality of sensors is disposed at predetermined locations along the belt body adapted to measure movements of the patient and anticipate movements of the patient. A control system is electronically coupled to the plurality of sensors and adapted to receive and interpret electronic signals sent from the sensors for calculating a movement value which can then be referenced against a preprogrammed value in the control system, and an active system is electronically coupled to the control system and adapted to adjust the size of the at least one conformable chamber to restrict anterior movement of the patient's vertebrae, wherein the active system is controlled by the control system.

Another aspect of the present invention includes an orthotic spinal support device for controlling movement of a patient, comprising an outer garment configured to be disposed about the lower torso of the patient in use. The support device further comprises an active system disposed on the outer garment having a plurality of active zones that are conformable in size and disposed adjacent a patient's abdomen in use and are adapted to restrict vertebral anterior movement of the patient by laterally applying pressure to the abdomen. A plurality of sensors are disposed at predetermined locations along the outer garment and adjacent to the patient for measuring movement, or the potential of movement, in the patient and further adapted to produce electronic signals containing movement or potential movement measurements. A control system is electronically coupled to the sensors and adapted to receive and interpret the electronic signals from the sensors for calculating a value which can then be referenced against a preprogrammed value in the control system, such that the control system can activate the active system to adjust the size of the plurality of active zones to restrict vertebral anterior movement of the patient.

These and other features, objects and advantages of the present invention will become apparent upon reading the following description thereof together with reference to the accompanying drawings.

### BRIEF DESCRIPTION OF THE DRAWINGS

FIG. 1 is a front planar view of an orthotic lumbar support belt of the present invention;

FIG. 2 is a rear planar view of the orthotic lumbar support belt of FIG. 1;

FIG. 3 is a perspective view of an orthotic lumbar support belt in a buckled condition;

FIG. 4 is a front elevational view of an orthotic lumbar support belt, shown as disposed on a skeletal reference;

FIG. 5 is a side elevational view of the lumbar support belt on a skeletal reference, as shown in FIG. 4;

FIG. 6 is a front elevational view of an orthotic lumbar support belt as disposed on a patient;

FIG. 7 is a front elevational view of an orthotic lumbar support belt as disposed on a patient;

FIG. 8 is a front elevational view of an orthotic lumbar support belt as disposed on a patient;

FIGS. 9A-9D are perspective views of orthotic lumbar devices according to additional embodiments of the present invention with particular reference to different fastening references disposed in the orthogonal support belt;

FIG. 10 is a front planar view of another embodiment of the present invention;

FIG. 10A is a front planar view of another embodiment of the present invention;

FIG. 11 is a perspective view of another embodiment of the present invention;

FIG. 11A is an exploded view of the device shown in FIG. 11;

FIG. 11B is an exploded view of another embodiment of the present invention;

FIG. 12 is a perspective view of another embodiment of the present invention;

FIG. 13 is a perspective view of another embodiment of the present invention;

FIG. 14 is a perspective view of another embodiment of the present invention having a control system;

FIG. 14A is a perspective view of another embodiment of the present invention having a wireless control system;

FIG. 15 is a block diagram depicting the operation of a control system;

FIG. 16 is a perspective view of another embodiment of the present invention; and

FIG. 16A is a perspective view of another embodiment of device shown in FIG. 16.

### DETAILED DESCRIPTION OF THE PREFERRED EMBODIMENT

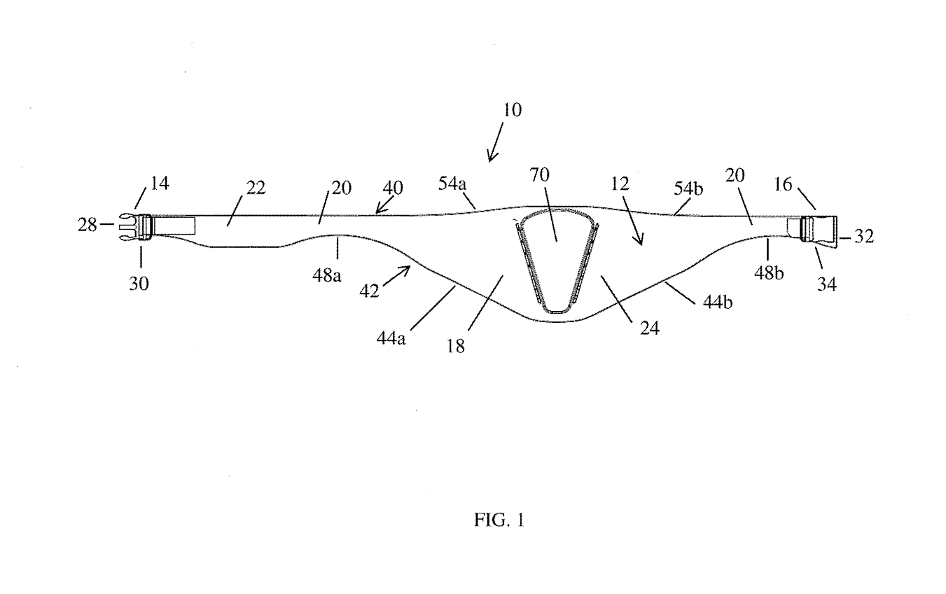

For the purposes of description herein, the terms “upper,” “lower,” “right,” “left,” “rear,” “front,” “vertical,” “horizontal,” and derivatives thereof shall relate to the invention as oriented in FIG. 1. However, it is to be understood that the invention may assume various alternative orientations, except where expressly specified to the contrary. It is also to be understood that the specific devices and processes illustrated in the attached drawings, and described in following specification, are simply exemplary embodiments. Hence, specific dimensions and other physical characteristics relating to the embodiments disclosed herein are not to be construed as limiting, unless expressly stated otherwise.

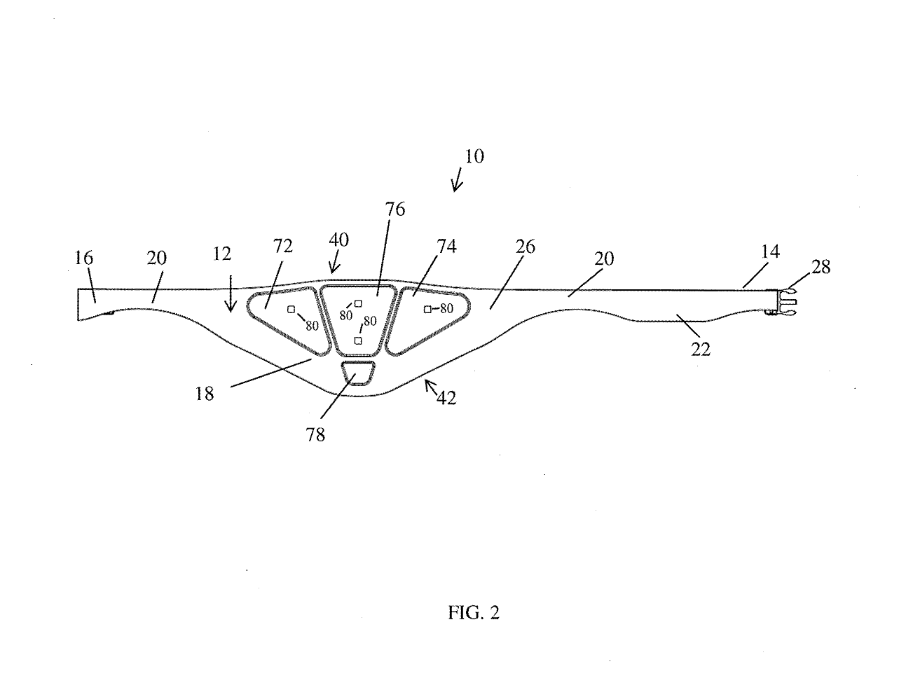

Referring to FIG. 1, the reference numeral 10 generally designates an orthotic garment or support device which, as shown in FIG. 1, is in the form of an orthotic lumbar support belt. The belt 10 generally comprises a belt-shaped body portion 12 having a first end 14 and a second end 16. The belt-shaped body portion 12 further includes a front portion or panel 18, side portions or panels 20, and a rear portion or panel 22. The belt-body 12 is generally configured as an outer shell having an A-side 24 (FIG. 1) and a B-side 26 (FIG. 2), wherein active zones, such as inflatable air chambers, can be disposed between the A-side 24 and B-side 26 of the belt body 12 as further described below.

As shown in FIG. 1, the first end 14 and second end 16 comprise fastener mechanisms, such as a male fastener 28 disposed at a terminal end of the belt body 12 adjacent first end 14, and a female fastener mechanism 32 disposed at a terminal end of the belt body 12 adjacent second end 16. In assembly, the male fastener 28 is adapted to operably couple to female fastener 32, such that the orthotic belt 10 can wrap around a patient to provide support for the patient as further described below. In the embodiment shown in FIG. 1, adjustment mechanisms 30, 34 are associated with the male and female fasteners 28, 32, which provide a source of adjustment to increase or decrease the circumference of the orthotic belt 10 when first end 14 is fastened to second end 16.

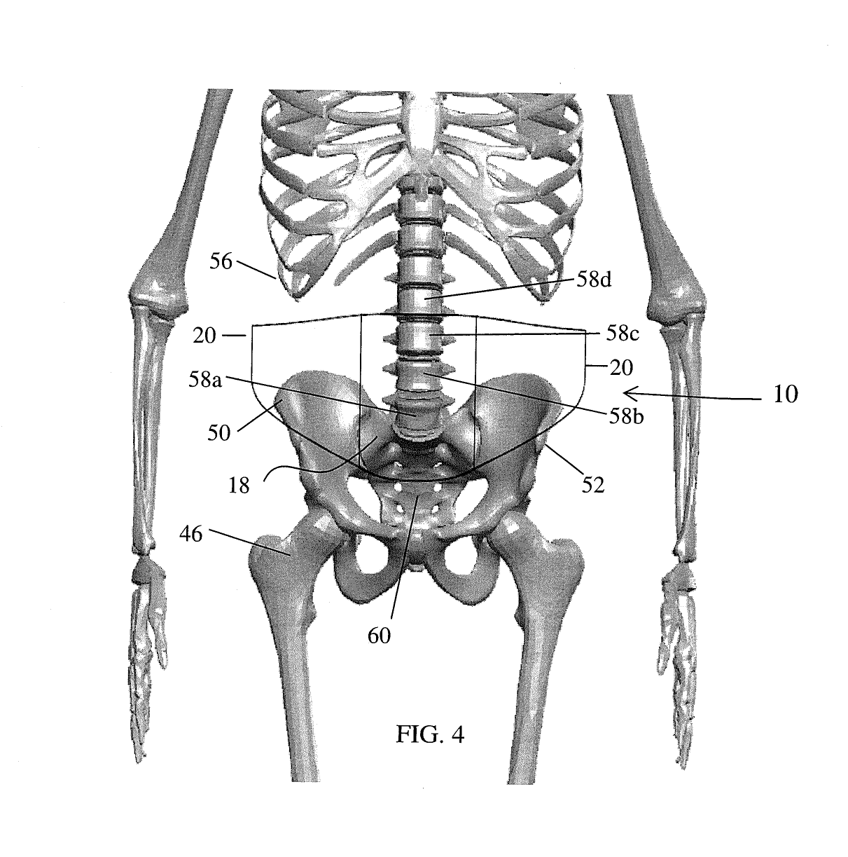

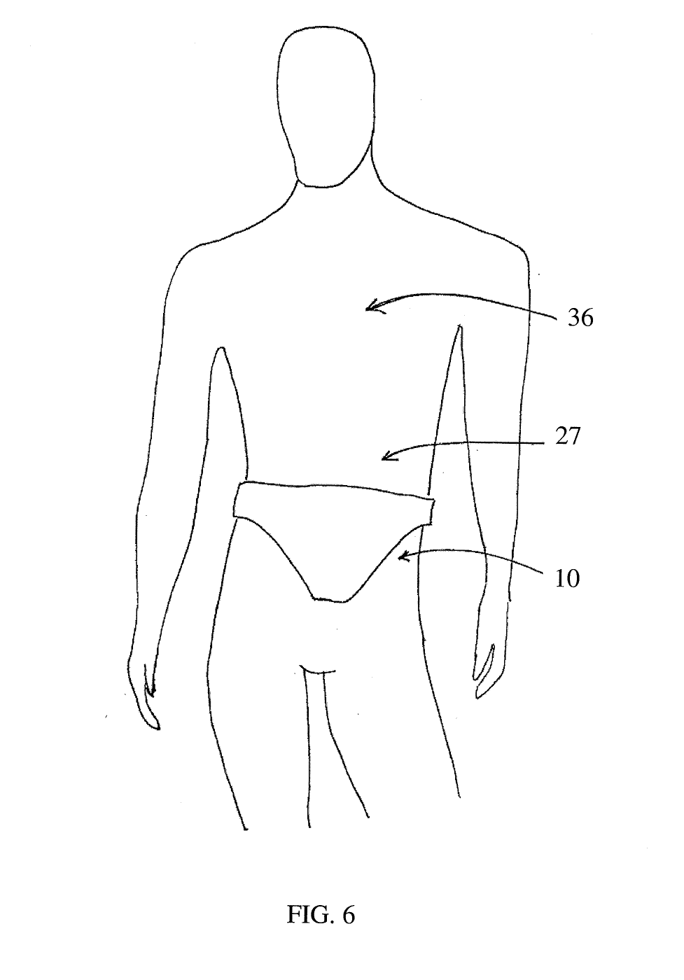

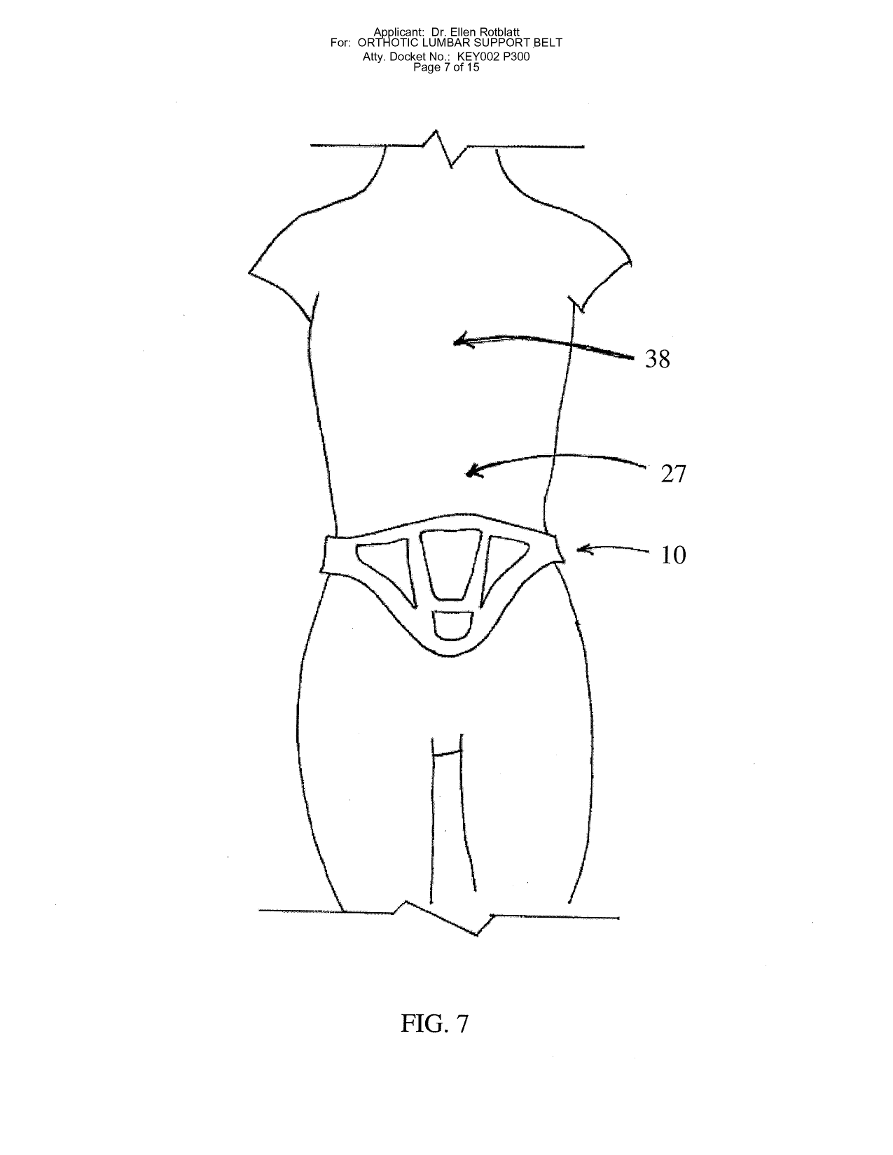

The orthotic lumbar support belt 10 is configured to be worn around the lower torso of a patient or user, as shown in FIGS. 4-8. The orthotic lumbar belt 10 can be specifically configured for use about a patient's lower torso 27 for use with a male user 36 (FIG. 6) or for use with a female user 38 (FIG. 7).

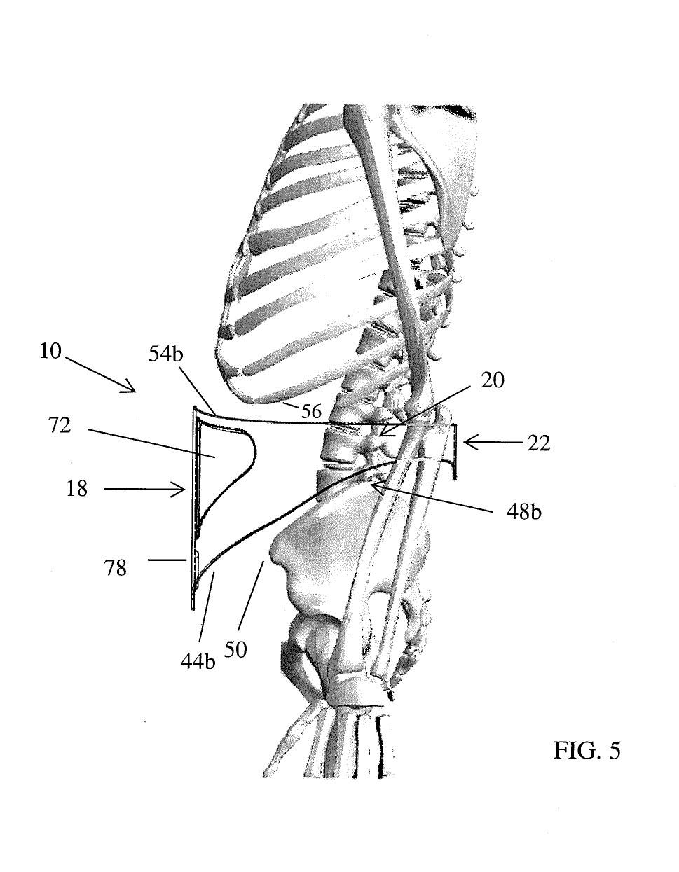

With reference to FIGS. 1 and 4, the orthotic lumbar support belt 10 comprises various contours disposed along the belt body 12 on both an upper side 40 and a lower side 42 of the belt body 12. Specifically, contours 44a and 44b are disposed on left and right sides of the front panel 18 and are configured to fit along the user's inguinal line, generally indicated by area 46, as shown in FIG. 4. Contours 48a and 48b are disposed adjacent 44a and 44b and are configured to form to the user's iliac crest, as generally indicated by the area designated with reference numeral 50 in FIG. 4. The contours 44a, 44b and 48a, 48b are designed to positionally situate the orthotic lumbar belt 10 at the user's anterior superior iliac spine, generally indicated by reference numeral 52 in FIG. 4. In this way, the contours 44a, 44b, 48a, 48b help to properly situate the orthotic lumbar belt 10 on the user and prevent rotation of the orthotic lumbar belt 10 around the user's lower torso. On the upper side 40 of the belt body 12, contours 54a, 54b are disposed on left and right sides of the front panel 18 and are configured to correlate to a lower portion of the user's rib cage, indicated by reference numeral 56 in FIG. 4. Thus, when the orthotic lumbar support belt 10 is positioned on the user, the rear panel 22 is disposed adjacent the user's lower spine, as shown in FIG. 5, and the front panel 18 is disposed about the user's lower torso or abdomen. Further, as shown in FIG. 5, the side panels 20 are disposed along a side of the user.

As shown in FIG. 4, side panels 20 are disposed above the iliac crest 50 of the user, and the front panel 18 is disposed along a vertebral column comprising the lumbar vertebra identified as reference numerals 58a-58d. While the front panel 18 appears larger than the rear panel 22 in the embodiment described thus far, it is contemplated that either the front panel 18 or the rear panel 22 can extend from the user's sacrum 60 to the lower portion of the user's rib cage 56, as required by the user.

With reference to FIGS. 1 and 2, the lumbar support belt 10 is shown in FIG. 1 with the exterior or A-side 24 depicted, whereas FIG. 2 depicts and interior or B-side 26. In the embodiment shown in FIGS. 1 and 2, the fastening mechanism comprising male fastener 28 and female fastener 32 is disposed on the belt body 12 in such a configuration that, when fastened about a user, the fastener will be disposed near a right side 20 on the user. The fastening mechanism depicted in FIG. 1 is a male-to-female clip mechanism; however, other fastening mechanisms are contemplated, such as a high load ski-boot type fastener mechanism, and other assemblies as further described below.

In the embodiment shown in FIGS. 1 and 2, the orthotic lumbar belt 10 comprises a control system 70, which is disposed on the A-side or exterior side 24 of the belt body 12 adjacent the front panel 18. The control system 70 is used to control a variety of active zones which, as shown in FIG. 2, are disposed on the interior or B-side 26 adjacent the front panel 18. The active zones in FIG. 2 include left and right active zones 72, 74, a central active zone 76, and a lower active zone 78. It is contemplated that the orthotic lumbar belt 10 can comprise any number of active zones in multiple configurations as necessitated by a patient's needs. Further, the active zones can be located at the sides 20 and the rear panel 22 of the orthotic lumbar belt 10 as well. Again, placement of the active zones is dictated by the spinal condition of the user, and the active zone placement shown in FIG. 2 is for exemplary purposes only. The active zones are adjustable zones which adjust in size to impart a motion resistant force on the user at the location of the active zone. In the embodiment shown in FIG. 2, the active zones 72, 74, 76, 78 would impart a lateral force on the user in a front-to-back direction, as indicated in FIG. 5 by arrow F, to restrict the movement of the patient's vertebrae in an anterior direction, as indicated by arrow A in FIG. 5.

As shown in FIG. 2, the active zones 72, 76, and 74 further comprise sensors 80 that are in electronic communication with the control system 70. In this way, the sensors 80 can send pressure, movement, position, and other like information to the control system 70, which can then vary the applied pressure of the active zones for adjusting the applied pressure according to a patient's needs. It is contemplated that the control system 70 can be programmed by a patient's doctor, or other healthcare professional, to regulate the amount of applied pressure from the active zones using data acquired from the sensors 80. Further, it is contemplated that microprocessors (not shown) can be incorporated into the control system 70 for active adjustment of the active zones according to movements of the patient. In this way, the present invention provides for active real time adjustability of the active zones according to the patient's movements. This information, with respect to the patient's movements and the applied pressure of the active zones as collected by the microprocessors and the sensors 80 can be stored in control system 70 and reviewed by the patient's doctor for analysis of the patient's on-going treatment. It is further contemplated that biometric chips can be incorporated into the control system 70 for housing patient identification information, as well as patient diagnosis information.

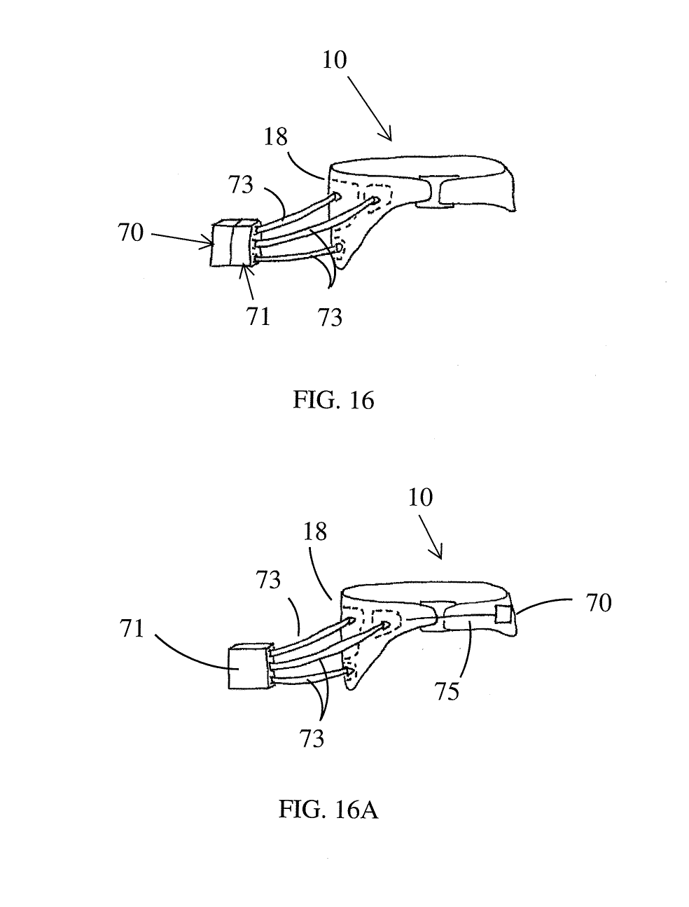

The active zones, such as active zones 72, 74, 76, and 78 shown in FIG. 2, are contemplated to be comprised of air chambers or bladders which can inflate or deflate using a pneumatic pump, such as pump 71 shown in FIGS. 16 and 16A, that is controlled by the control system 70 in reference to pressure sensing data collected by the sensors 80 disposed on or near the active zones. It is further contemplated that the orthotic lumbar belt 10 of the present invention can incorporate a manual pump for active adjustment of the active zones by the patient. While the active zones contemplated in FIG. 2 are described as air bladders or air chambers, it is contemplated that any number of pressure applying active zones can be used with the present invention to restrict patient movement as further described below.

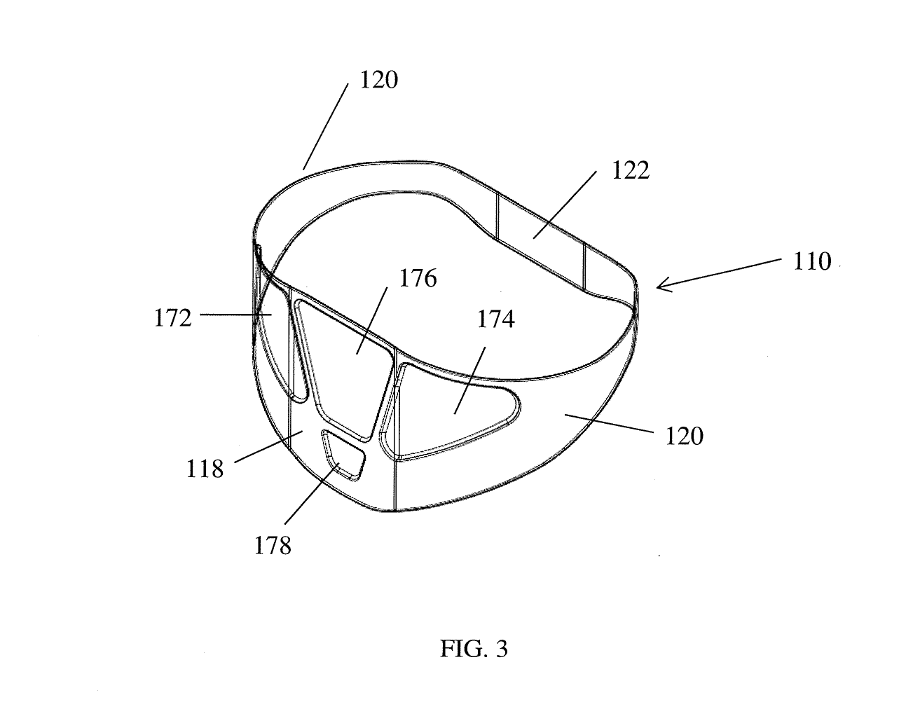

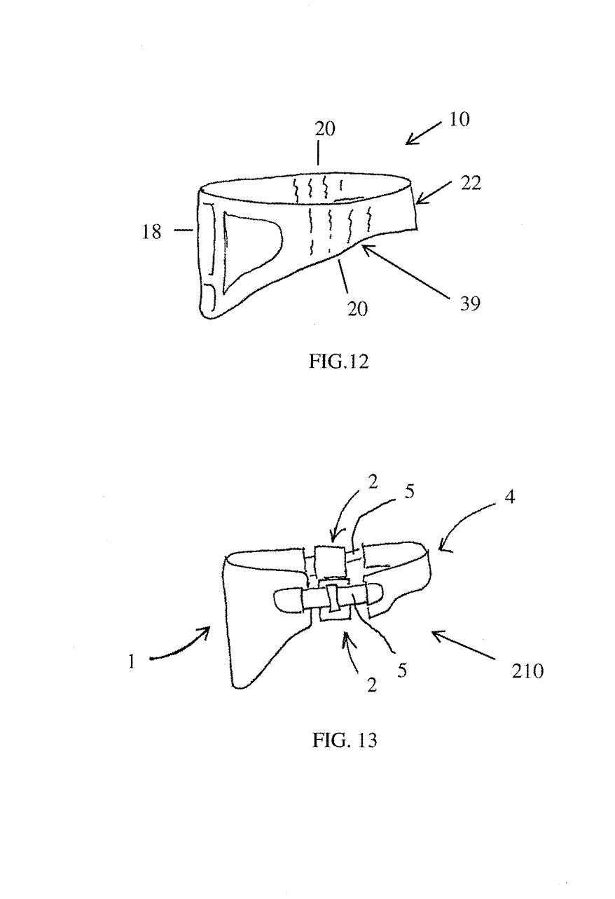

Referring now to FIG. 3, another embodiment of an orthotic lumbar belt is shown, as indicated by reference numeral 110, having a front panel 118, a rear panel 122, and side panels 120. The orthotic lumbar belt 110 of FIG. 3 comprises a belt body 112 on which the front, rear, and side panels are disposed, and the belt body 112 is a singular unit providing a continuous shell for housing active zone recesses 172, 174, 176, and 178 adjacent the front panel 118 in a similar configuration as the embodiment shown in FIGS. 1 and 2, wherein the recesses are adapted to house active zones in assembly. Thus, it is contemplated that the orthotic lumbar belt of the present invention can be a continuous one-piece unit, such as orthotic lumbar belt 110 (FIG. 3), or belt 10 shown in FIG. 12, where a fastening mechanism is not necessary, or the orthotic lumbar belt can be a modular orthotic lumbar belt, such as the modular orthotic lumbar belt 210, shown in FIG. 13 having discontinuous side panels 2, a front panel 1, and a rear panel 4, which, in the embodiment of FIG. 13, are interconnected by adjustable or flexible bands 5.

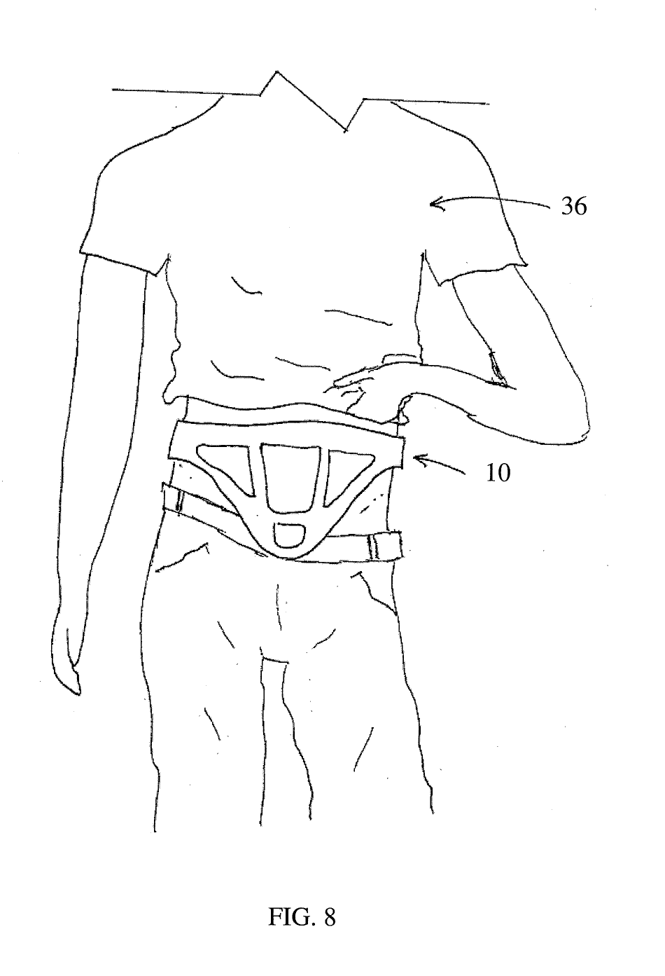

As shown in FIG. 8, the orthotic lumbar belt 10 can be worn in an inconspicuous manner under a patient's clothing. In this way, the orthotic lumbar belt 10 can be worn by the user 36 at all times in a concealed manner, such that the user is not discouraged from using the orthotic lumbar belt 10 when necessary.

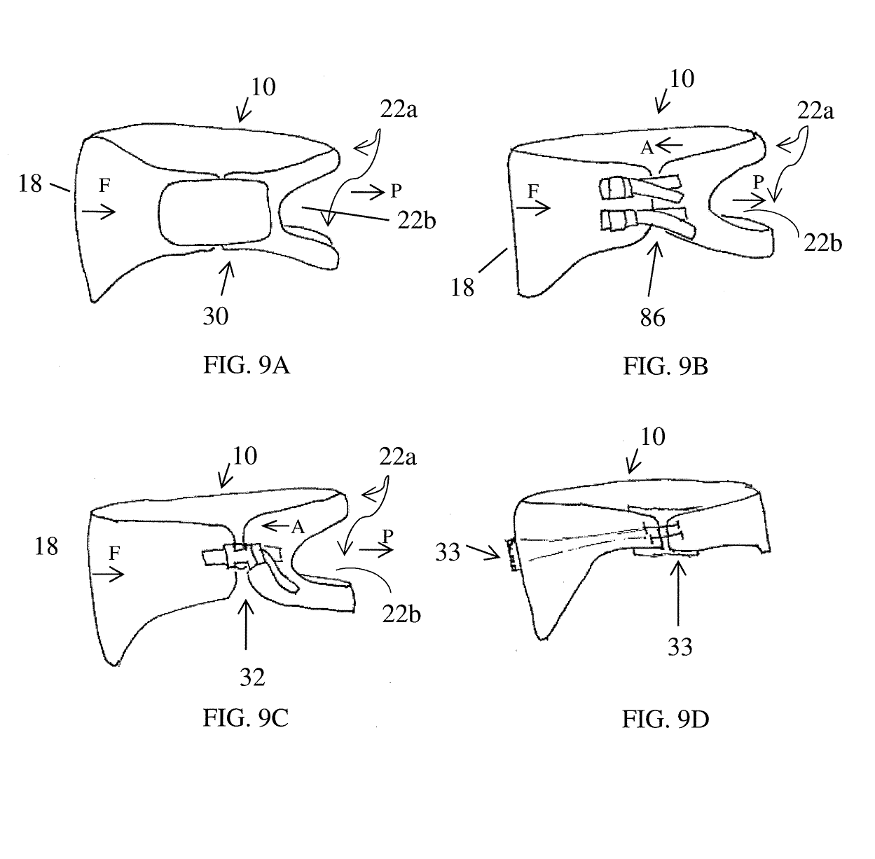

FIGS. 9A-9D indicate various embodiments of the orthotic lumbar belt of the present invention. Specifically, FIGS. 9A-9C show the rear panel being a two-part strap system, as indicated by reference numeral 22a. Further, FIGS. 9A-9D indicate various fastener mechanisms that can be incorporated into the present invention. Specifically, FIG. 9A indicates a hook-and-loop fastening system designated by reference numeral 84. Reference numeral 86 in FIG. 9B indicates belts or straps 31 used as a fastening mechanism on the orthotic lumbar belt 10. A quick-snap fastener 88 is depicted in FIG. 9C, and a ratcheting mechanism 90 is shown in FIG. 9D as another embodiment of a fastening mechanism for the orthotic lumbar belt 10. Specifically, ratcheting mechanism 90 of FIG. 9D can have several different attachment points for ratcheting together various sections of the device about a user. While the fastening mechanisms of the orthotic lumbar belt allow for adjustability of the overall circumference of the orthotic lumbar belt, the overall sizing of the orthotic lumbar belt 10 is determined during a doctor patient sizing process. The slight adjustability in the fastening mechanism of the orthotic lumbar belt is included to allow for adjustability in light of unforeseen circumstances where adjustability is needed for increased patient comfort.

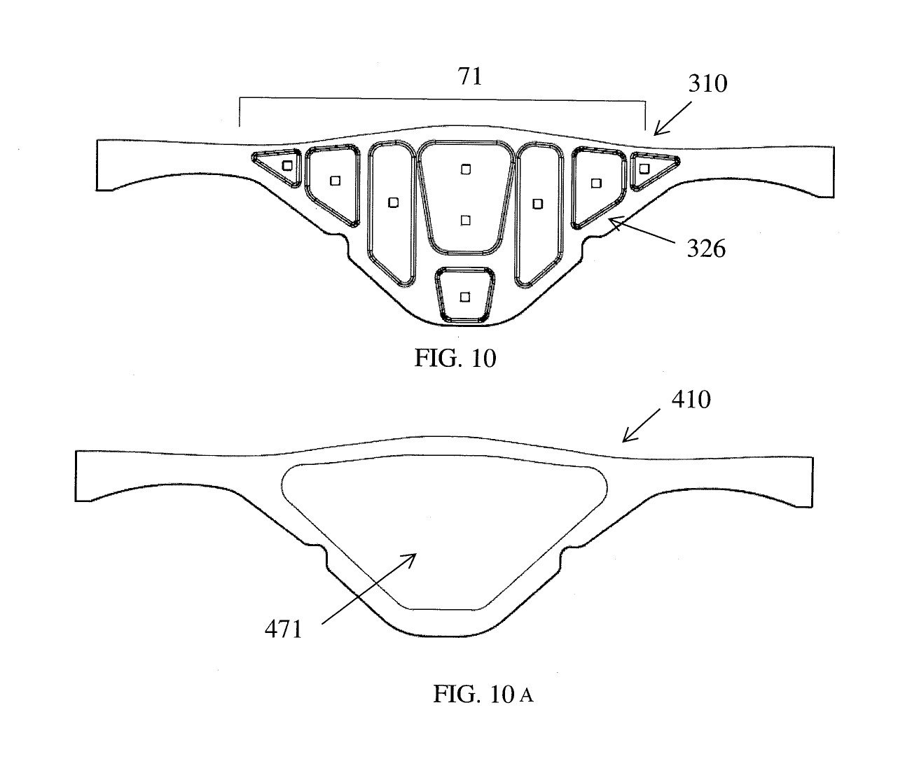

As shown in FIGS. 10 and 10A, another embodiment of the present invention is indicted by reference numeral 310, wherein the orthotic lumbar belt 310 includes a plurality of active zones as generally indicated by reference numeral 371. FIG. 10 depicts and interior side or B-side 326 of the orthotic lumbar belt 310.

FIG. 10A depicts another embodiment of the present invention, as indicated by reference numeral 410, having a single active zone 471 in a similar configuration as the plurality of active zones 371 shown in FIG. 10.

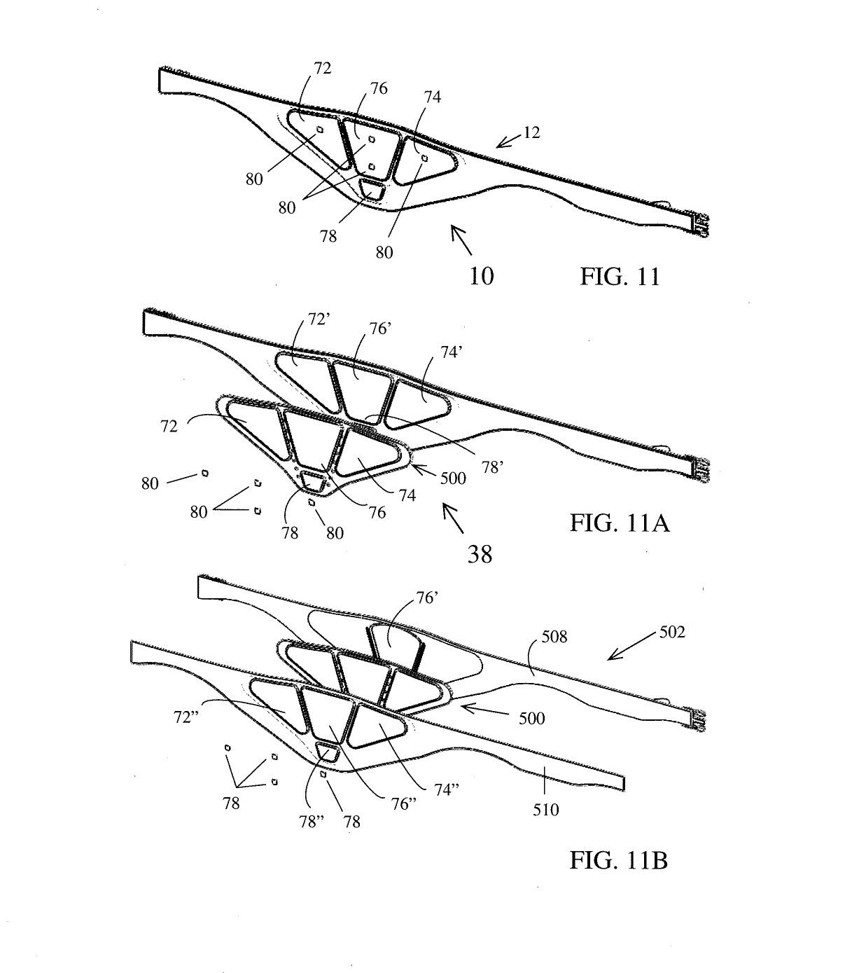

As shown in FIGS. 11-11A, an embodiment of the present invention is shown as indicated by reference numeral 10, similar to the embodiment shown in FIGS. 1 and 2 as described above. In FIG. 11A, the belt 12 is shown with the active zones 72, 74, 76 and 78, as well as sensors 80 exploded away from the belt 12 to reveal recesses 72′, 74′, 76′ and 78′ which correlate to the active zones 72, 74, 76 and 78 which are shown in FIG. 11A disposed on an active zone panel 500. Referring to FIG. 11B, another embodiment of the present invention is shown having a belt apparatus comprising an A-side 508 and a B-side 510 with an active zone panel 500, having active zones like the panel shown in FIG. 11A disposed thereon, wherein the active zone panel 500 is disposed between the A-side 508 and the B-side 510 in assembly. The embodiment 502 shown in FIG. 11B further comprises recesses 72″, 74″, 76″ and 78″ on the B-side 510 which are adapted to house the active zones of the active zone panel 500.

For descriptive purposes regarding the operation of the orthotic lumbar support device of the present invention, the device will be parceled out according to subsystems of the invention including a passive system, an active system and a control system. In practice the orthotic lumbar support device could be configured so that it consists of one, two or all three of the subsystems noted above. Furthermore, the invention could be configured so that all three systems are integrated together as a unitary whole, such as the orthotic lumbar belt 10 shown in FIGS. 1 and 2, or, the device could be configured in a modular configuration, such as the orthotic lumbar belt 210 shown in FIG. 13 wherein the subsystems are independent of one another. With reference to FIGS. 1 and 2, the passive system of the orthotic lumbar belt 10 contains the main supports of the device, such as the belt body 12, which gives the overall garment its shape, and further houses the control system 70 and the active system comprising active zones 72, 74, 76 and 78. The passive system is constructed of generally flexible fabric materials, and reinforcements, such as rigid rib members and the like, can be added in areas which are intended to be stationary or require increased rigidity in use.

With reference to FIGS. 1 and 2, a large front panel 18 is located at the front of the device 10 and is disposed adjacent the user's abdomen in use. The front panel 18 is generally centered between a user's Anterior Superior Iliac Spine (ASIS). The shape of the device 10 comprises contours, such as contours 44a, 44b, 48a and 48b, such that the device 10 is contoured to fit along the patient's Inguinal Line (inner thigh) 46 and Iliac Crests (hips) 50 (FIG. 5) for retention of the device 10 in a proper position and for increased patient comfort when in use.

As described above, the front panel houses various active zones (72, 74, 76 and 78) which make up the active system of the device 10. Additional active zones can be located in the side panels 20 as well as the rear panel 22 as necessitated by an individual patient's needs. The front panel 18 further comprises recesses or cavities which house the active zones in assembly.

FIG. 3 is an exemplary embodiment of a device 110 indicating a series of recesses 172, 174, 176 and 178 disposed in a front panel 118 for housing active zones of a specific configuration. The shape and configuration of the active zones can vary as shown in FIGS. 2 and 10.

The side panels 20 of the device 10 are generally more rigid then the front panel 18. The side panels 20 can also accommodate various active zones or simply be constructed of more rigid materials based upon the necessities of the patent, such as reinforcement ribs 39 shown in FIG. 12. The side panels 20 of the device 10 are worn above the user's hip bones and are contoured for comfort. As noted above, with reference to FIG. 13, the side panels 2 of another embodiment of the belt device 210 can be modular, or they can be integrated into the front or rear panels as shown in FIGS. 1 and 2. It is further contemplated that the side panels can be fully integrated into both the front and rear panels.

The rear panel 22 of the belt 10 consists of one panel (FIG. 1) or multiple panels 22a (FIGS. 9B and 9C) attached to the sides panels 20 and generally located on a patient's spinal column in use. The rear panels could cover between the T10 (thoracic) vertebrae and sacrum depending on the belts configuration and the needs of the patient. Rigidity of the rear panel's can also vary depending on the needs of the patient. As shown in FIGS. 9A-9C the rear panel comprises two straps 22a disposed above and below a gap 22b. The gap or aperture 22b allows for the vertebrae of the patient to move laterally in posterior direction as indicated by the arrow P. The posterior movement P of the patient's vertebrae is encouraged in the embodiments having a gap or aperture 22b (FIGS. 9A-9C), and this configuration further ensures that anterior movement, as indicated by arrow A, of the patient's vertebrae is restricted as controlled by the force, indicated by arrow F, emanating from the front panel 18 by active zones disposed thereon.

The overall passive system consists of various configurations or modules to accommodate a patient's gender, body shape, and medical condition. Once configured for a patient the device 10 is designed to be mildly adjustable to accommodate weight gain, swelling and such. However, the adjustability after initial fitting is limited to prevent the patient from injuring themselves or reducing the effectiveness of the system. The device 10 (FIG. 1) has a belt body portion 12 having first and second ends 14, 16 that are joined together via a connection system, such as fasteners 28, 32, preferably located adjacent the front panel 18 or side panel 20 of the belt 10. This connection system allows for the complete removal of the device 10 from the user.

The active system of the design consists of a series of active zones, which, as shown in the embodiment of FIGS. 1 and 2, includes active zones 72, 74, 76 and 78. These active zones are designed to increase or decrease resistance to bending and/or twisting to limit a patient's mobility in use. The active system can be computer controlled by a control system 70 or be adjusted manually by pneumatic pumps or other such devices. In a computer controlled system, the active zones will self adjust and will continuously adjust based upon the patient's actions. The active zones could be mechanical, electrical, pneumatic or hydraulic and housed within the passive system. The active zones can be directly integrated into the passive belt or be modular and removable as shown in FIG. 11B.

The size, shape, number, and locations of the active zones will vary based upon the patient's needs. In one embodiment, shown in FIG. 2, four active zones are included in the belt assembly 10. The largest active zone 76 is placed directly in the middle of the front panel 18 so as to be adjacent to the abdominal wall of the patient in use. The placement of active zone 76 centered at the naval of the patient is designed to prevent forward flexure or anterior movement of the patient's spine. Two zones 72, 74 are placed in a flanking position of the large central zone 76. These active zones 72, 74 cover the abdomen between the crest of the hip and the center of the abdominals and are designed to prevent flexure at 45 degree angles. A fourth and smallest, active zone 78 is located directly beneath the large central zone 76. The purpose of this lower zone 78 is to prevent extreme flexure such as the flexure that occurs in a patient in a fetal position. Additional smaller and more numerous zones can be positioned in the same areas to prevent additional degrees of motion and give the device a higher degree of adaptability, such as the active zone configuration 371 shown in the device 310 of the embodiment of FIG. 10.

The control system 70 of the present invention consists of an onboard computer controller and a series of sensors 80. The sensors 80 are positioned throughout the belt 10 and their purpose is to determine the speed, direction, and potential of motion of the patient. The computer controller is integrated into the control system and is adapted to monitor information relayed from the sensors 80 as the control system is operably coupled to the sensors. In response to information relayed from the sensors 80, the control system will send a signal to the active system to increase or decrease the resistance in the various active zones found on the belt body 12. The sensors 80 could be accelerometers, potentiometers, and Electromyography (EMG) type sensors. Other sensors, such as position sensors, temperature sensors, pressure transducers and the like can be used as dictated by the needs of the patient. The sensors 80 are contemplated to be positioned in areas such as key muscle groups and anatomical locations such as the ASIS as necessary for properly monitoring a patient's movements.

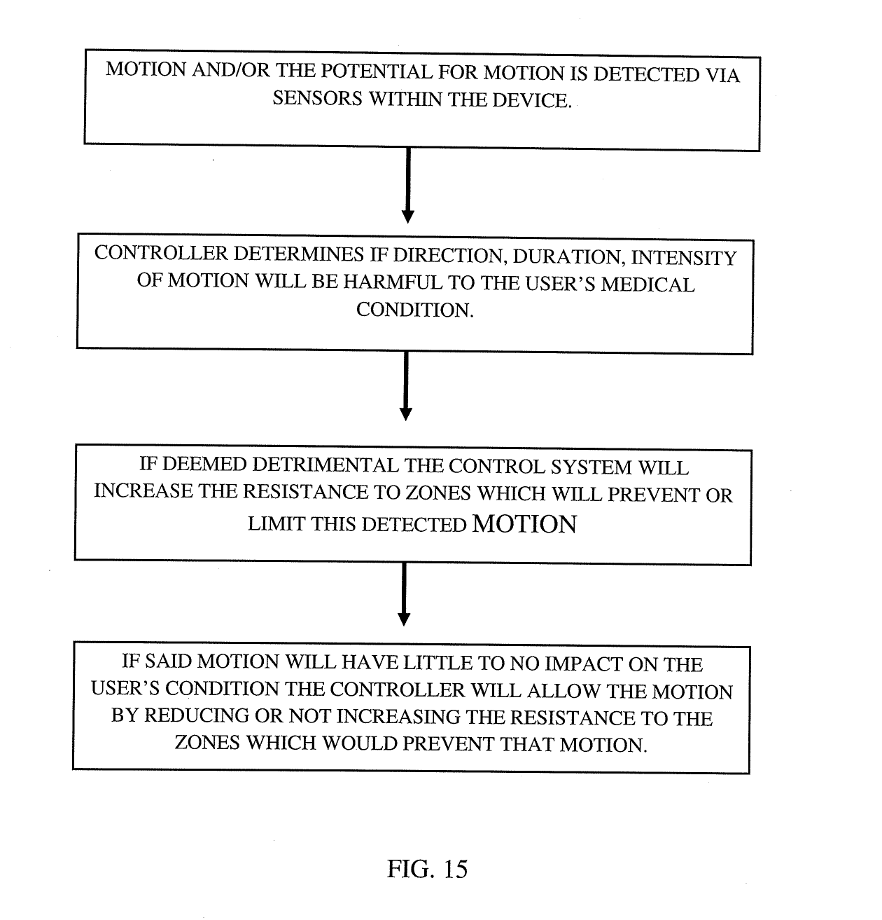

Thus, as shown in the block diagram of FIG. 15, the systems of the device 10 cooperate to monitor and control a patient's movement. The sensors 80 are positioned within the orthotic belt 10 with the purpose of the detecting motion or the potential of motion within a patient. The control system 70 interprets whether or not the current motion or anticipated motion will be harmful to the patient. If actual motion is detected, the control system 70 will use information from the sensors 80 to determine the direction, duration and intensity of the movement. The control system 70 will then vary the amount of resistance applied by the active zones, 72, 74, 76 and 78, in accordance with the orthotic's programming which is prep-programmed into the control system by a healthcare professional at the initial device fitting. The programming incorporated into the device 10 houses information regarding the user's medical needs and current medical conditions, and the control system 70 uses this information to either allow, restrict, or prevent a given motion that is either occurring or anticipated.

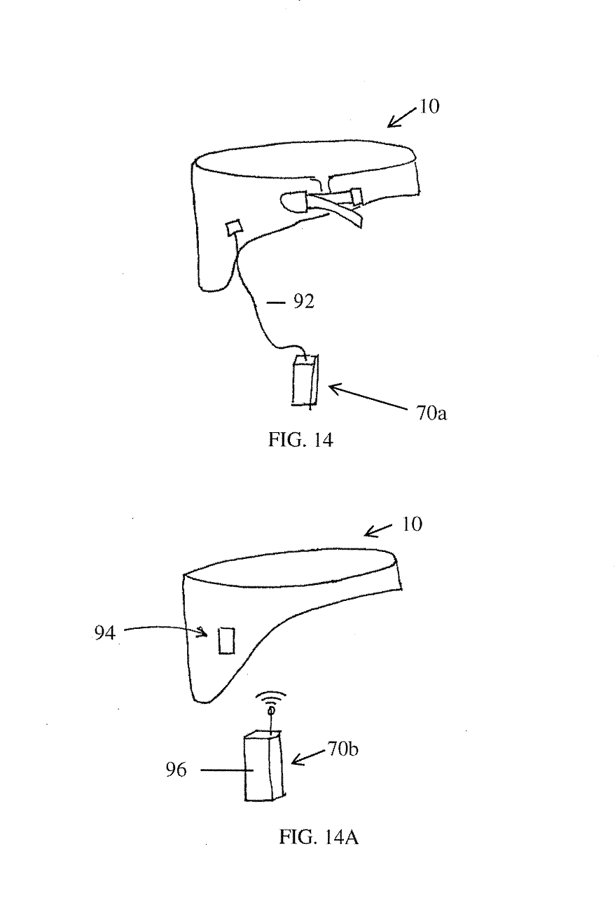

The control system 70 could be located on the front panel 18 of the device, such as shown in FIG. 1, or in any other location along the belt body 12 of the device 10 that is conveniently accessible to the patient. Another embodiment of a control system 70a could be located externally of the orthotic 10 via a tether 92 (FIG. 14) such that a user of the device 10 could easily control the active zones of the device 10 while the device 10 is concealed under the user's clothing. The orthotic 10 could also be configured with another embodiment of a control system 70b wherein a receiver 94 is disposed within the belt body 12 of the device 10 which can wirelessly control the active zones by a hand held remote control 96.

Generally, in operation, the orthotic lumbar support device of the present invention is designed uniquely address spinal conditions of a patient such as spondylolithesis (LS), spondylosis, a slipped disk condition and other similar conditions. The key feature of the present invention is that it is a reverse mechanism for the treatment of LS as compared to other known orthotic support mechanisms. The present invention is essentially “reversed” in nature in that it can provide support from the front of the patient towards the back of the patient as indicated by arrow F in FIG. 5. The support given by the device in the direction of arrow F ensures that the patient's vertebrae do not move in the anterior direction as indicated by arrow A. The resistance applied by the active zones of the device 10 is not only customizable, but prescribeable by a healthcare professional, such that the support provided by the orthotic lumbar belt 10 of the present invention is specifically tailored to the end-user during an initial fitting. Further, unlike other support mechanisms, the support provided by the active zones of the present invention is active in that it may change according to a healthcare professionals programming based on the input received by the control system from the customized placement of various sensors 80 disposed on the device 10.

As noted above, the present invention relates to an orthotic lumbar support garment intended to be worn under a patient's clothing and affixed around the abdomen of the patient generally positioned above the patient's hips. Designed in a one-piece construction with variable widths dependent on the patients morphological anatomy, the orthotic lumbar support garment is positioned in a level manner around the mid to lower abdominal region, such that the caudal aspect rides just above the pelvic crest on the lateral sides, and the pubic symphysis on the anterior region. The orthotic lumbar support garment of the present invention is in the form of a belt-like apparatus with both the hardware and software of the device having been formatted and pre-programmed for the individual patient at the time of evaluation by a doctor or other healthcare provider. To best configure the parameter limits of expansion of the active zones according the patient's degree of spinal impairment, the device programming and fitting is conducted during the patient evaluation. Factors such as the patient's habits, activity level are factored into the fitting and programming of the device. The software is configured to be actively working in a 3-dimensional axis, instantly reacting with infinite gradations of air bladder filling or active zone expansion. The very dynamic active zone expression that correlates to a patient's intended motion is based on real-time data collected by the plurality of sensors which analyze such things as force of motion, acceleration deceleration, as well as motion vectors in a three dimensional setting. If any sensor or combination of sensors detects an upcoming or in-progress motion which would aggravate various spinal conditions existing in a patient, the dynamic interior then serves to either prevent, restrict or reduce this upcoming or intended motion. Spinal conditions existing in a patient, such as spondylolithesis (LS), spondylosis, a herniated or dessicated disc, when aggravated may cause varying degrees of sciatica which is an excruciatingly debilitating condition causing unrelenting pain to radiate down the buttocks to one or both of the lower extremities. This is most often ultimately due to lumbar foraminal spinal stenosis.

As noted above, patients currently in need of spinal braces or other orthopedic devices have very limited options for a non-surgical treatment. The physician may voice his or her preferences for the patient, or the patient may attempt, without any understanding of the etiology causing his/her debility, to ask for particular features which rarely have any utility regarding their lumbar spine disease state. Devices are available currently which are either custom-made for the individual patient, or are themselves customizable, but both options have significant limitations as compared to the dynamic real-time active zone response of the present invention. As stated above, a customizable orthotic device is a device which uses pre-manufactured modular components that allow a patient, for whom has no medical knowledge of what is causing the pain, to customize the device according to their individual needs without supervision. This type of orthotic device, if not immediately discontinued, has a significant risk of worsening the patient's debilitating condition to the degree of a de-stabilization which is then a surgical emergency. As noted above, the customizable device, being a pre-manufactured device without the individual characteristics of the patient being considered, can lead not only to an ill-fitting device, but a device which is directly destructive depending on which of the customizable features and in what combination these static features are used.

The present invention provides a dynamic, not static, orthopedic lumbar device which is professionally fitted and programmed using state of the art micro-electronic sensors that can be pre-programmed to different degrees of three dimensional motion restriction. The orthotic lumbar support device instantly reacts long before what is sensed to be a hazardous patient movemetn has gotten very far underway. A particular spinal diagnosis in one person may require very different parameters and sensitivities of settings form patient to patient with the same or similar diagnosis. The non-invasive dynamic orthotic lumbar support device of the present invention is intended and manufactured for one particular patient in accordance with the specific medical condition(s) of that patient. Thus, the present orthotic lumbar support device offers superior comfort and increased medical benefit to the patient via quality of life, reversal to an asymptomatic state, and at the very least, a degree of prophylaxis with maintenance of stability allowing the patient to carry on a more normal life-style than they had ever been previously allowed with presently known non-invasive support devices.

Generally, traditional spinal orthopedic devices are still designed to be static and severely limit a patient's motion in most, if not all, directions, regardless of the spinal condition of the patient. These known devices do not discriminate between motions which are harmful to the patient's condition and motions which, in fact, would be beneficial to the patient. The vector, duration, rotational axis, degree of acceleration or deceleration of various motions of a patient are dictated by the software formatting of the patient's spinal condition, and are reviewed for any necessary changes at the time of the patient's next physician's appointment.

The present invention is considered a smart orthopedic garment in the form of an orthotic lumbar support belt which comprises an outer shell, having an infinitely dynamic and adaptive subsystem. The garment must be properly fitted for any corrective activation to occur, and thus, has built-in safety release mechanisms as well to relieve pressure when release is indicated as necessary.

The present invention includes an orthotic spinal support device for controlling movement of a patient, comprising a belt-like apparatus having a relatively flat portion made of a material general more rigid in nature than the materials used on the lateral aspects of the device. The present invention functions to disperse and displace any force exceeding pre-set and preprogrammed limits in any direction which could further harm a patient suffering from a given spinal condition. Surprisingly, an unforeseen utility of the present invention is to assist in the teaching of muscle memory in a patient by controlling the patient's posture. The lumbar region designed to be treated with the orthotic of the present invention is exceedingly more at risk from both internal and external forces going from anterior to posterior. The belt apparatus includes multiple interconnected conformable chambers or bladders configured to apply multi-directional pressure, stabilizing predetermined areas of lumbar spine of the patient to limit anterior movement, acceleration or deceleration, or lateral rotation exceeding prescribed limits of motion.

The plurality of sensors disposed at predetermined locations along the belt body of the present invention are adapted to measure real-time movements of the patient as well as anticipate the patient immediately pending movements by means of building electrical sensor charges which can be processed by the controller. The sensors of the present invention can be low-voltage sensors, or, more preferably, ultra-low voltage sensors. Incorporating ultra-low voltage sensors in the present invention allows for the present invention to have a much more compact, slim-line, low profile power module. This power pack can be inserted into an external or mid portion of the thickness of the belt body of the orthotic. Insertion into a pocket on either lateral side of the orthotic is preferable.

As noted above, a data controller is electronically coupled to the plurality of sensors and adapted to receive and interpret electronic signals sent from the sensors for calculating all movement values which can then be referenced against preprogrammed values in the control system, and an active system is electronically coupled to the control system and adapted to adjust the size of the at least one conformable chamber to restrict anterior movement of the patient's vertebrae, wherein the active system is controlled by the control system.

As shown in FIG. 16A, a data controller 70 is electronically coupled to sensors 80 and adapted to receive and interpret electronic signals sent from the sensors 80 through wire runners 75 coupled to both the sensors 80 and the controller 70 for calculating all movement values which can then be referenced against preprogrammed values stored in the controller 70 and set by the patient's healthcare provider during an office evaluation. An active system is shown disposed on a front panel 18 of the orthotic device 10 which is electronically coupled to the control system or controller 70. The control system or controller 70 is adapted control a pump assembly 71 which is coupled to the individual active zones disposed on the front panel 18 of the device 10 by transport runners 73 designed to carry liquid or air or any other suitable material capable of controlling the size of the active zones. In operation, the pump 71 is able to adjust the size of the active zones which comprise conformable chambers. The load produced by the increased size of the active zones is adapted to restrict anterior movement of the patient's vertebrae, unlike the anterior directional loads created by orthotic devices known in the art. As shown in FIG. 16, the controller 70 and the pump 71 are integrated into one modular unit coupled to the orthotic device 10.

It will become apparent to those skilled in the art that various modifications to the preferred embodiment of the invention as described herein can be made without departing from the spirit or scope of the invention as defined by the appended claims.

The invention claimed is:

1. An orthotic spinal support device for controlling movement of a patient, comprising:
a belt apparatus having a belt body portion adapted to couple to a patient adjacent a spinal region of the patient, the belt apparatus comprising;
at least one conformable chamber configured to apply lateral pressure to predetermined areas of an abdomen of the patient to limit anterior movement of a patient's vertebrae;
a plurality of sensors disposed at predetermined locations along the belt body adapted to measure movements of the patient and anticipate movements of the patient;
a control system electronically coupled to the plurality of sensors and adapted to receive and interpret electronic signals sent from the sensors for calculating a movement value which can then be referenced against a preprogrammed value in the control system; and
an active system electronically coupled to the control system and adapted to adjust the size of the at least one conformable chamber to restrict anterior movement of the patient's vertebrae, wherein the active system is controlled by the control system.

2. The orthotic spinal support device as defined in claim 1, wherein the belt apparatus further comprises a front portion, a rear portion and side portions.

3. The orthotic spinal support device as defined in claim 1, wherein the plurality of sensors includes sensors selected from the group consisting of accelerometers, pressure sensors, temperature sensors, pressure transducers, and location sensors.

4. The orthotic spinal support device as defined in claim 1, wherein the active system comprises a pump operably coupled to the at least one conformable chamber, and the control system comprises controls for adjusting the size of the at least one conformable chamber by adjusting a volume of the conformable chamber.

5. The orthotic spinal support device as defined in claim 3, wherein the pump is a pneumatic pump adapted to control an airflow into and out of the at least one conformable chamber.

6. The orthotic spinal support device as defined in claim 3, wherein the pump is a liquid pump adapted to control an amount of liquid into and out of the at least one conformable chamber.

7. The orthotic spinal support device as defined in claim 2, wherein the at least one conformable chamber is disposed on the front portion of the belt apparatus.

8. The orthotic spinal support device as defined in claim 2, wherein the rear portion comprises a gap disposed adjacent to the patient's vertebrae and adapted to allow posterior movement of the patient's vertebrae.

9. The orthotic spinal support device as defined in claim 8, wherein the rear portion further comprises support straps disposed above and below the gap.

10. An orthotic spinal support device for controlling movement of a patient, comprising:
an outer garment configured to be disposed about the lower torso of the patient in use;
an active system disposed on the outer garment comprising a plurality of active zones that are conformable in size and disposed adjacent a patient's abdomen in use and further adapted to restrict vertebral anterior movement of the patient by laterally applying pressure to the abdomen;
a plurality of sensors disposed at predetermined locations along the outer garment and adjacent to the patient for measuring movement or the potential of movement in the patient and further adapted to produce electronic signals containing movement or potential movement measurements; and
a control system electronically coupled to the sensors and adapted to receive and interpret the electronic signals from the sensors for calculating a value which can then be referenced against a preprogrammed value in the control system, such that the control system can activate the active system to adjust the size of the plurality of active zones to restrict vertebral anterior movement of the patient.

11. The orthotic spinal support device as defined in claim 10, wherein the outer garment is configured to wrap around a patient and further comprises a front portion, a rear portion and side portions with a releasable fastening mechanism.

12. The orthotic spinal support device as defined in claim 11, wherein the outer the front portion is disposed adjacent the patient's abdomen in use and further houses the active system.

13. The orthotic spinal support device as defined in claim 12, further comprising a second active system disposed in anyone of the portions selected from the group consisting of the rear and side portions.

14. The orthotic spinal support device as defined in claim 12, further comprising contours disposed on opposite sides of the front portion and adapted to conform to an inguinal line of the patient.

15. The orthotic spinal support device as defined in claim 12, wherein the plurality of sensors includes sensors selected from the group consisting of accelerometers, pressure sensors, temperature sensors, pressure transducers, and location sensors.

16. The orthotic spinal support device as defined in claim 12, further comprising a pump adapted to adjust the size of the active zones, wherein the pump is controlled by the control system.
